(19) United States
(12) Patent Application Publication
(10) Pub. No.: US 2008/0051461 A1
(43) Pub. Date: Feb. 28, 2008

(54) PEELING PROCESS BASED ON SURFACTANTS AND ACIDS

(75) Inventors: Odile AUBRUN-SONNEVILLE, Antony (FR); Michelle Rathman Josserand, La Celle St Cloud (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/842,342

(22) Filed: Aug. 21, 2007

Related U.S. Application Data
(60) Provisional application No. 60/840,957, filed on Aug. 30, 2006.

(30) Foreign Application Priority Data
Aug. 23, 2006 (FR) 06 53429

(51) Int. Cl. A61K 31/60; A61Q 19/00

(52) U.S. Cl. 514/568

(57) ABSTRACT

Process for the treatment of human skin, by applying topically onto the skin a composition containing in a physiologically acceptable medium (i) at least one hydroxy acid selected from the α-hydroxy acids, β-hydroxy acids, α-keto acids, β-keto acids and mixtures thereof, and (ii) at least 5% by weight, relative to the total weight of the composition, of one or more surfactants containing an alkyl chain having from 6 to 16 carbon atoms.

### REFERENCE TO PRIOR APPLICATIONS

This application claims priority to U.S. provisional application 60/840,957 filed Aug. 30, 2006, and to French patent application 0653429 filed Aug. 23, 2006, both incorporated herein by reference.

### FIELD OF THE INVENTION

The present invention relates to a peeling process which makes use of a composition containing at least 5% of surfactants containing an alkyl chain having from 6 to 16 carbon atoms and at least one hydroxy acid.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Peeling is a well-known means for improving the appearance of the surface of the skin, in particular for treating visible and/or tactile irregularities of the human skin, and for example to attenuate defects of pigmentation such as skin freckles or the marks due to acne or varicella, or to smooth irregularities in the texture of the skin, in particular wrinkles or minor wrinkles.

This peeling has the effect of removing part of the skin to be treated (epidermis and possibly upper layer of dermis) by chemical methods such as the application of compositions containing high concentrations of agents stimulating the desquamation of the skin, such as hydroxy acids such as glycolic acid or salicylic acid, or else other active substances such as retinoic acid, resorcinol, trichloroacetic acid or phenol. Thus, the document U.S. Pat. No. 6,787,148 describes compositions containing anhydrides containing a phenol and a derivative of polyethylene glycol.

Increasing concentrations of active substance and in particular of hydroxy acids make it possible to increase the efficacy of the products. However, such concentrations result in substantial problems during application and after application (reddening, tingling, burning sensation). Thus, peeling using salicylic acid can give rise to cases of salicylate poisoning in case of overdosage or prolonged application.

### SUMMARY OF THE INVENTION

There is a need therefore for available peeling compositions which are effective while being better tolerated.

The inventors have discovered that a combination of at least one hydroxy acid and at least 5% by weight of one or more surfactants comprising at least one alkyl chain having from 6 to 16 carbon atoms provide peeling compositions that are simultaneously effective and well tolerated.

Nothing in the prior art suggested that surfactants having a C6-C16 alkyl chain used at such a concentration could be combined with, e.g., AHA or BHA for the creation of chemical peeling agents that are both effective and better tolerated.

The document U.S. Pat. No. 5,939,085 discloses a process for smoothing the skin with exfoliating particles such as polyethylene particles. Such smoothing process differs from peeling by the fact that smoothing acts at the surface of the skin in order to remove dead skins and smoothing the skin, but it does not allow to treat irregularities of the skin, such as wrinkles and marks due to acne. Furthermore, this smoothing process is made with physical exfoliating agent (polyethylene particles) while peeling is made with chemical agents such as hydroxy acids used in great amount. In the invention peeling process, preferably no exfoliating particles are used.

### DETAILED DESCRIPTION OF THE PREFERRED EMBODIMENTS

One aspect of the invention is a process for the treatment of visible and/or tactile irregularities of the human skin, comprising:

(a) applying topically onto the skin a composition comprising in a physiologically acceptable medium (i) at least one hydroxy acid selected from the α-hydroxy acids, β-hydroxy acids, α-keto acids, β-keto acids and mixtures thereof, and (ii) at least 5% by weight, relative to the total weight of the composition, of one or more surfactants comprising an alkyl chain having from 6 to 16 carbon atoms, the total quantity of hydroxy acids and surfactants comprising an alkyl chain having from 6 to 16 carbon atoms being at least 15% by weight relative to the total weight of the composition.

As used herein, a surfactant comprising an alkyl chain having from 6 to 16 carbon atoms includes a surfactant having more than one alkyl chain having from 6 to 16 carbon atoms.

The process also optionally comprises:

(b) optionally leaving the composition in contact with the skin for prolonged time, and
(c) optionally removing the composition by rinsing.

The composition preferably does not include exfoliating particles such as polyethylene particles.

Preferably, the composition is left in contact with the skin for an application time which is sufficient for the composition to act. This time will vary depending on the concentration of surfactants and hydroxy acids in the composition and on the effect desired. As a guide, the composition can remain in contact with the skin or the integuments for a period of a minute or two, 5 minutes, etc., generally lying between 5 minutes and 12 hours, preferably between 5 minutes and 6 hours, preferably between 5 minutes and 30 minutes. The composition may or may not be removed at the end of this contact time. Application can be daily or twice daily, or more, or weekly, and repeated at intervals of, e.g., from 2 weeks to 6 months, it being possible to prolong or repeat this period without difficulty.

As the composition is preferably intended for topical application, it contains a physiologically acceptable medium. “Physiologically acceptable medium” is understood to mean a medium compatible with keratinous materials such as the skin, the lips, the nails, the scalp and/or the hair. The composition is in particular a cosmetic or dermatological composition, more particularly intended for peeling of the skin.

The medium is an aqueous medium, in other words it contains water the quantity whereof is preferably at least 15% by weight even better at least 20% by weight relative to the total weight of the composition. The quantity of water can for example range from 15 to 85% by weight or more, preferably from 20 to 80% by weight, better from 25 to 75% by weight and still better from 30 to 70% by weight relative to the total weight of the composition.

The composition preferably has a pH which can range from 1 to 9, preferably from 1 to 5 and better from 1.5 to 5.

### Surfactants

The composition according to the invention comprises at least 5% by weight of one or more surfactants containing at least one alkyl chain having from 6 to 16 carbon atoms, and preferably from 6 to 14 carbon atoms.

The surfactant or mixture of surfactants preferably has (have) an HLB (Hydrophilic Lipophilic Balance) greater than 8, preferably an HLB of at least 9. Surfactants having an HLB less than or equal to 8 can also be used, especially when one or more other surfactants are added thereto so that the HLB of the mixture is greater than 8.

The surfactants used according to the present invention can be selected from the nonionic, anionic, cationic, amphoteric or zwitterionic surfactants, and mixtures thereof. They can be obtained from alcohols, acids, amines, amides, alkyl glycerols and from any group containing at least one alkyl chain ranging from C6 to C16, preferably from C6 to C14.

The polar part of these surfactants can be nonionic, anionic, cationic and amphoteric or zwitterionic.

The nonionic surfactants can for example be selected from surfactants containing a group selected for example from the polyalkylene glycol groups such as in particular polyethylene glycol or polypropylene glycol, polyglycerol groups, sugar groups (glucose, maltose, sorbitol, ethoxylated sorbitan), and mixtures thereof.

The anionic surfactants can for example be selected from surfactants containing a group selected for example from the sulphate, sulphonate and phosphate groups, amino acid such as glycinate, glutamate and derivatives of these amino acids, in particular salts thereof, and mixtures thereof.

The cationic surfactants can for example be selected from surfactants containing for example a quaternary ammonium group.

The amphoteric or zwitterionic surfactants can for example be selected from surfactants containing for example the amphoacetate, amphodiacetate, betaine or sultaine groups.

The composition can contain a mixture of these different sorts of surfactants.

When the composition has a pH less than or equal to 2, the surfactants used in the composition of the invention preferably do not contain an ester or amide bond.

As nonionic surfactants, for example the following can be cited:
alkylpolyglycosides and in particular alkylpolyglucosides (APG) having an alkyl group containing from 6 to 16 carbon atoms (C6-C16 alkyl polyglucosides) and preferably 8 to 16 carbon atoms, such as for example decylglucoside (C9/C11 alkyl (1.4)-polyglucoside) such as the product marketed under the name MYDOL 10 by the company Kao Chemicals, The product marketed under the name PLANTAREN 2000 UP or PLANTACARE 2000 UP by the company Cognis, and the product marketed under the name ORAMIX NS 10 by the company Seppic, caprylyl/capryl glucoside such as the product marketed under the name ORAMIX CG 110 by the company Seppic, laurylglucoside such as the products marketed under the names PLANTAREN 1200 N and PLANTACARE 1200 by the company Cognis and coco-glucoside such as the product marketed under the name PLANTACARE 818/UP by the company Cognis;
esters of polyethylene glycol and acids containing at least one alkyl chain ranging from C6 to C16, preferably from C6 to C14, such as polyethylene glycol (8 EO) myristate such as the product marketed by the company Gattefosse under the name of MIRLENE,
derivatives of polyethylene glycol and of mono-, di- and triglycerides of acid(s) containing at least one alkyl chain ranging from C6 to C16, preferably from C6 to C14, and having at least two ethylene oxide groups, and preferably from 6 to 8 ethylene oxide groups, such the mono-, di- and triglycerides of caprylic acid and capric acid, such as that containing 6 ethylene oxide groups (INCI name: PEG-6 caprylic/capric glycerides), marketed under the name SOFTIGEN 767 by the company Sasol, that containing 8 ethylene oxide groups (INCI name: PEG-8 caprylic/capric glycerides), marketed under the name L.A.S. by the company Gattefosse, and that containing 7 ethylene oxide groups, marketed under the name CETIOL HE 810 by the company Cognis (INCI name: PEG-7 Caprylic/Capric Glycerides) and the ethoxylated (20 EO) glyceryl monolaurate marketed under the name TAGAT L 2 by the company Degussa-Goldschmidt;
ethoxylated derivatives of esters of sorbitan and acid(s) containing at least one alkyl chain ranging from C6 to C16, preferably from C6 to C14, such as the PEG-10 Sorbitan Laurate marketed under the name LIPOSORB L10 by the company Lipo Chemicals;
sugar esters containing at least one C6 to C16 alkyl chain, such as the mixture of sucrose laurate and sucrose dilaurate, marketed by the company Mitsubishi Chemical under the name SURFHOPE SE COSME C-1216;
esters of polyglycerol and acid(s) containing at least one C6 to C16 alkyl chain, such as the polyglyceryl monolaurate (10 moles of glyceryl) (INCI name: Polyglyceryl-10 Laurate) marketed by the company Sakamoto Yakuhin under the name S FACE L-1001;
polyglycerol ethers such as the polyglycerol-3 hydroxylauryl ether produced by Chimex under the name Chimexane NF;
and mixtures thereof.

Preferably, the composition is free from ethers of polyethylene glycol and alcohols such as the Laureth.

As anionic surfactants, the following can for example be cited:
alkylsulphates, alkyl ether sulphates and salts thereof, in particular sodium salts thereof, such as sodium lauryl ether sulphate such as the product marketed under the name TEXAPON AOS 225 UP by the company Cognis;
monoalkyl and dialkyl esters of phosphoric acid and salts thereof, such as for example sodium mono- and dilauryl phosphate, potassium mono- and dilauryl phosphate, triethanolamine mono- and dilauryl phosphate, sodium mono- and dimyristyl phosphate, potassium mono- and dimyristyl phosphate, diethanolamine mono- and dimyristyl phosphate, and triethanolamine mono- and dimyristyl phosphate;
derivatives of amino acids, in particular alkali salts of amino acids, such as:
acylsarcosinates such as the sodium lauroyl sarcosinate marketed under the name SARKOSYL NL 97® by the company Ciba or marketed under the name ORAMIX L 30® by the company Seppic, the sodium myristoyl sarcosinate marketed under the name NIKKOL SARCOSINATE MN® by the company Nikkol, and the sodium palmitoyl sarcosinate marketed under the name NIKKOL SARCOSINATE PN® by the company Nikkol,
acyl alaninates such as the sodium N-lauroyl-N methyl amidopropionate marketed under the name SODIUM NIKKOL ALANINATE LN 30® by the company Nikkol, or marketed under the name ALANONE ALE® by the company Kawaken and the triethanolamine N-lauroyl N-methyl alanine marketed under the name ALANONE ALTA® by the company Kawaken,
acylglutamates such as the triethanolamine mono-cocoyl glutamate marketed under the name ACYLGLUTAMATE CT-12® by the company Ajinomoto, and the triethanolamine lauroylglutamate marketed under the name ACYLGLUTAMATE LT-12® by the company Ajinomoto, and
acylglycinates such as the sodium N-cocoyl glycinate marketed under the names AMILITE GCS-12® and AMILITE GCK 12 by the company Ajinomoto.
alkyl ether carboxylates in particular those of formula:

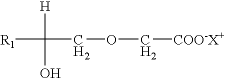

wherein:

### R1 more particularly represents a saturated or unsaturated, linear or branched alkyl group containing from 8 to 16 carbon atoms,

X represents hydrogen or an inorganic or organic cation such as those derived from an alkali metal (for example Na+ or K+), NH4+, ammonium ions derived from basic amino acids such as lysine, arginine, sarcosine, ornithine, citrulline or else amino alcohols such as monoethanolamine, diethanolamine, triethanolamine, glucamine, N-methyl glucamine and 3-amino-1,2-propanediol. Preferred 2-hydroxy alkyl ether carboxylic acids are compounds of the formula (I) wherein R1 more particularly represents a mixture of C8-C16 groups, in particular derived from copra. Among the surfactants of formula (I), the product marketed under the name BEAULIGHT SHAA by the company SANYO may in particular be cited;
and mixtures thereof.

The cationic surfactants utilisable according to the present invention can in particular be salts of primary, secondary or tertiary amines, containing one C6 to C16 alkyl chain, which may be polyalkoxylated, quaternary ammonium salts, imidazoline derivatives or amine oxides of a cationic nature.

In the quaternary ammonium salts, the anion is preferably a halide (chloride, bromide or iodide) or an alkylsulphate, more particularly a methylsulphate. However, quaternary ammonium salts wherein the anion is a methanesulphonate, a phosphate, a nitrate, a tosylate, an anion derived from an organic acid such as acetate or lactate or any other anion compatible with the ammonium ion with ester group can be utilized. Still more particularly, the anion is chloride or methylsulphate.

Among the quaternary ammonium salts, the following can in particular be cited:
tetraalkylammonium chlorides such as for example dialkyldimethylammonium or alkyltrimethylammonium chlorides, wherein the alkyl group contains from about 6 to 16 carbon atoms, such as for example the dodecyl trimethyl ammonium chloride marketed under the name ARQUAD 12-50 by the company Akzo Nobel, or the cetyl trimethyl ammonium chloride marketed under the name DEHYQUART A OR by the company Cognis;
quaternary ammonium salts containing at least one ester group, such as for example the dicocoylethyl hydroxyethyl methyl ammonium methosulphate marketed under the name DEHYQUART L 80 by the company Cognis;
quaternary ammonium salts containing a sugar moiety, for example a glucose, fructose or saccharose moiety, such as for example the Butyldimoniumhydroxypropyl Laurylglucosides chloride (INCI name: Butyldimoniumhydroxypropyl Laurylglucosides Chloride) marketed under the name Colonial SugaQuat TM-1212 by the company Colonial Chemical Inc, and the Lauryl Methyl Gluceth-10 Hydroxypropyldimonium chloride (INCI name: Lauryl Methyl Gluceth-10 Hydroxypropyldimonium Chloride) marketed under the name GLUCQUAT 125 by the company Noveon.

The amphoteric and zwitterionic surfactants can for example be selected from the derivatives of betaine, and in particular the alkyl derivatives of betaine, the alkylamidopropylbetaines, alkylamphoacetates, hydroxy-sultaines, and mixtures thereof.

As betaine derivatives, in particular alkylbetaines containing a C6-C16, and more particularly C6-C14, alkyl group and ethoxylated derivatives thereof of these alkylbetaines, for example cocobetaine such as the product marketed under the name DEHYTON AB-30® by the company Cognis, laurylbetaine such as the product marketed under the name GENAGEN KB® by the company Clariant, ethoxylated (10 EO) laurylbetaine, such as the product marketed under the name LAURYLETHER(10 EO)BETAINE® by the company Shin Nihon Rica, and ethoxylated (10 EO) stearylbetaine such as the product marketed under the name STEARYLETHER(10 EO)BETAINE® by the company Shin Nihon Rica can be cited.

As alkylamidopropylbetaines, for example C6-C16 alkyl-amidopropyl betaines such as the cocamidopropyl betaine marketed for example under the name VELVETEX BK 35® by Cognis or else the undecylenamidopropyl betaine marketed for example under the name AMPHORAM U® by Ceca can be cited.

As alkylamphoacetates, for example C6-C16 alkyl-amphoacetates such as N-disodium N-cocoyl-N-carboxy-methoxyethyl-N-carboxy-methylethylenediamine (INCI name: disodium cocamphodiacetate) such as the product marketed under the name MIRANOL C2M CONCENTRE NP® by the company Rhodia Chimie, and N-sodium N-cocoyl-N-hydroxyethyl-N-carboxymethylethylenediamine (INCI name: sodium cocamphoacetate).

The composition utilized in the process according to the present invention comprises a quantity of surfactant(s), which can be adjusted in accordance with the depth of the peeling which has to be performed. This quantity is at least 5% by weight and preferably at least 10% by weight relative to the total weight of the composition. It can range from 5 to 70% by weight, preferably from 10 to 60% by weight and better from 15 to 50% by weight relative to the total weight of the composition.

### Hydroxy Acids

The composition according to the invention comprises one or more hydroxy acids selected from the α-hydroxy acids, β-hydroxy acids, α-keto acids, β-keto acids and mixtures thereof. According to a preferred mode of implementation of the invention, the hydroxy acids are selected from the α-hydroxy acids and the α-keto acids, and mixtures thereof.

More particularly, the following can be cited as examples of α-hydroxy acids: citric acid, lactic acid, glycolic acid, tartaric acid, malic acid, mandelic acid, methyllacetic acid, glucuronic acid, pyruvic acid, phenyllactic acid, gluconic acid, galacturonic acid and mixtures thereof.

More particularly the following can be cited as examples of β-hydroxy acids: salicylic aid and derivatives thereof, in particular derivatives of the following formula (I) or a salt of such a derivative:

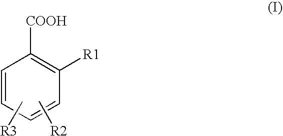

wherein:
R₁ represents a hydroxyl group or an ester of formula:

—O—CO—R4

wherein R₄ is a saturated or unsaturated aliphatic group, containing from 1 to 26 carbon atoms, and preferably from 1 to 18 carbon atoms, an amine or thiol group which may be substituted with an alkyl group containing from 1 to 18 carbon atoms, and preferably from 1 to 12 carbon atoms,
R₂ and R₃ independently of one another are located at position 3, 4, 5 or 6 on the benzene nucleus and independently of one another represent a hydrogen atom or a group:

—(O)ₙ—(CO)ₘ—R₅

wherein n and m, independently of one another, are each a whole number equal to 0 or 1, on condition that R₂ and R₃ are not simultaneously hydrogen atoms;
R₅ represents a hydrogen atom, a linear, branched or cyclized saturated aliphatic group containing from 1 to 18 carbon atoms, an unsaturated group containing from 3 to 18 carbon atoms, bearing one to nine conjugated or non-conjugated double bonds, it being possible for the groups to be substituted with at least one substituent selected from halogen atoms (fluorine, chlorine, bromine or iodine), trifluoromethyl groups, hydroxyl groups in free form or esterified with an acid containing from 1 to 6 carbon atoms, or carboxyl groups, free or esterified with a lower alcohol containing from 1 to 6 carbon atoms.

Preferably, the salicylic acid derivative of formula (I) is such that R₁ represents a hydroxyl group, R₂ represents a hydrogen atom and R₃ is in the 5 position of the benzene nucleus and represents a —CO—R₅ group where R₅ represents a saturated aliphatic group containing from 3 to 15 carbon atoms.

According to a preferred mode of implementation of the invention, the salicylic acid derivative of formula (I) is selected from 5-n-octanoylsalicylic, 5-n-decanoyl-salicylic, 5-n-dodecanoylsalicylic, 5-n-octylsalicylic, 5-n-heptyloxysalicylic, 4-n-heptyloxysalicylic, 5-tert-octylsalicylic, 3-tert-butyl-5-methylsalicylic, 3-tert-butyl-6-methylsalicylic, 3,5-diisopropylsalicylic, 5-butoxysalicylic, 5-octyloxysalicylic, propanoyl-5-salicylic, 5-n-hexadecanoyl-5-salicylic, 5-n-oleoyl-salicylic and 5-benzoylsalicylic acids, monovalent and divalent salts thereof and mixtures thereof. More particularly, it is 5-n-octanoylsalicylic (INCI name: Capryloyl salicylic Acid).

More particularly, the following can be cited as examples of α-keto acids: ascorbic acid (or vitamin C), and the ascorbyl glucosides. “Ascorbyl glucoside” is understood to mean the product of condensation of glucose, in the D form, that is to say in the form of α or β glucopyranose or α or β furanose, or in the L form, with ascorbic acid, preferably in the L form. More specifically, L-ascorbic acid 2-O-α-D-glucopyranoside (INCI name: Ascorbyl Glucoside), available from the company Hayashibara, can be cited.

The quantity of hydroxy acid(s) used depends on the desired aim. It can for example range from 5 to 70% by weight, preferably from 10 to 70% by weight and better from 10 to 60% by weight and still better from 10 to 50% by weight relative to the total weight of the composition.

According to the invention, the total quantity of hydroxy acids and surfactants containing one alkyl chain having from 6 to 16 carbon atoms is at least 15% by weight, preferably at least 20% by weight, better at least 25% by weight and still better at least 30% by weight relative to the total weight of the composition. This total quantity can range for example from 15 to 85% by weight, preferably from 20 to 80% by weight, better from 25 to 75% by weight and still better from 30 to 70% by weight relative to the total weight of the composition.

The composition according to the invention can be applied by any way enabling a uniform distribution on the skin, and in particular with the aid of fingers, cotton wool, a rod, a brush, a gauze, a spatula or a pad, or else by powdering, and it may or may not be removed. It can for example be removed by rinsing with water or simple wiping.

The compositions according to the invention can take any form, including all the galenical forms normally utilized in the cosmetic and dermatological fields, including in particular in the form of aqueous gels, lotions or emulsions. These compositions are prepared in accordance with the usual methods. According to a preferred mode of implementation of the invention, the composition takes the form of an aqueous, hydroalcoholic or hydroglycolic gel, or an aqueous, alcoholic or hydroglycolic solution.

When the composition according to the invention contains an oily phase, in particular when it is in the form of an emulsion, the oily phase preferably contains at least one oil, in particular a physiologically acceptable oil. It can further contain other fatty substances.

As oils utilizable in the composition of the invention, the following can for example be cited:
hydrocarbon oils of animal origin, such as perhydrosqualene;
hydrocarbon oils of plant origin, such as liquid triglycerides of fatty acids containing from 4 to 10 carbon atoms such as triglycerides of heptanoic or octanoic acids, or else for example sunflower, maize, soya, marrow, grape seed, sesame, hazel nut, apricot, macadamia, arara, castor or avocado oils, triglycerides of caprylic/capric acids such as those sold by the company Stearineries Dubois or those sold under the names Miglyol 810, 812 and 818 by the company Dynamit Nobel, jojoba oil, and shea butter oil;
synthetic esters and ethers, in particular of fatty acids, such as the oils of formulae R¹COOR² and R¹OR², wherein R¹ represents the residue of a fatty acid containing from 8 to 29 carbon atoms, and R² represents a branched or unbranched hydrocarbon chain, containing from 3 to 30 carbon atoms, such as for example Purcellin oil, isononyl isononanoate, isopropyl myristate, 2-ethylhexyl palmitate, 2-octyldodecyl stearate, 2-octyldodecyl erucate, isostearyl isostearate, isocetyl stearate, hydroxylated esters such as isostearyl lactate, octyl hydroxystearate, octyldodecyl hydroxystearate, diisostearyl malate, triisocetyl citrate, the heptanoates, octanoates and decanoates of fatty alcohols, polyol esters, such as propylene glycol dioctanoate, neopentyl glycol diheptanoate and diethylene glycol diisononanoate, and esters of pentaerythritol such as pentaerythrityl tetraisostearate;
linear or branched hydrocarbons, of mineral or synthetic origin, such as volatile or nonvolatile paraffin oils, and derivatives thereof, vaseline, polydecenes, hydrogenated polyisobutene such as parleam oil;
fatty alcohols having from 8 to 26 carbon atoms, such as cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleic alcohol;
partly hydrocarbon and/or silicone fluorinated oils such as those described in the document JP-A-2-295912;
silicone oils such as the volatile or nonvolatile polymethylsiloxanes (PDMS) with linear or cyclic silicone chain, liquid or pasty at ambient temperature, in particular cyclopolydimethylsiloxanes (cyclo-methicones) such as cyclohexasiloxane; polydimethyl-siloxanes containing alkyl, alkoxy or phenyl groups, within or at the end of the silicone chain, groups having from 2 to 24 carbon atoms; phenylated silicones such as the phenyltrimethicones, phenyldimethicones, phenyltrimethylsiloxydiphenylsiloxanes, diphenyl-dimethicones, diphenylmethyldiphenyl trisiloxanes, 2-phenylethyltrimethylsiloxysilicates, and polymethyl-phenylsiloxanes;
and mixtures thereof.

In the list of oils cited above, “hydrocarbon oil” is understood to mean any oil mainly containing carbon and hydrogen atoms, and possibly ester, ether, fluorinated, carboxylic acid and/or alcohol groups.

The other fatty substances which can be present in the oily phase are for example fatty acids containing from 8 to 30 carbon atoms, such as stearic acid, lauric acid, palmitic and oleic acid, waxes such as lanolin, beeswax, Carnauba or Candellila wax, paraffin or lignite waxes or microcrystalline waxes, ceresine or ozokerite, synthetic waxes such as polyethylene waxes, Fischer-Tropsch waxes, silicone resins such as trifluoromethyl-C1-4 alkyldimethicone and trifluoro-propyldimethicone, and silicone elastomers such as the products marketed under the names “KSG” by the company Shin-Etsu, under the names “Trefil”, “BY29” or “EPSX” by the company Dow Corning or under the names “Gransil” by the company Grant Industries.

These fatty substances can be selected in various ways by the person skilled in the art in order to prepare a composition having the desired properties, for example of consistency or texture.

When the composition is in the form of an emulsion, it is preferably a water-in-oil (O/W) emulsion. The emulsions generally contain at least one emulsifier selected in particular from the amphoteric, anionic, cationic or nonionic emulsifiers, used alone or mixed. They are preferably nonionic emulsifiers. These emulsifiers are selected from those typically used in the cosmetic field.

Emulsions can also be prepared without emulsifying surfactants or with a content thereof of less than 0.5% of the total weight of the composition, by using appropriate compounds, for example polymers having emulsifying properties such as the polymers marketed under the names Carbopol 1342 and Pemulen by the company Noveon, or the polymers and copolymers of 2-acrylamido 2-methylpropane sulphonic acid, which may be crosslinked and/or neutralized, such as the poly-(2-acrylamido 2-methylpropane sulphonic acid) marketed by the company CLARIANT under the name “Hostacerin AMPS” (INCI name: ammonium polyacryldimethyltauramide) or such as the emulsion polymer marketed under the name Sepigel 305 by the company Seppic (INCI name: Polyacrylamide/C13-C14 isoparaffin/laureth-7), particles of ionic or nonionic polymers, more particularly particles of anionic polymer such as in particular polymers of isophthalic acid or sulphoisophthalic acid, and in particular phthalate/sulphoisophthalate/glycol copolymers (for example diethylene glycol/phthalate/isophthalate/1,4-cyclohexane-dimethanol (INCI name: Diglycol/CHDM/isophthalates/SIP Copolymer) sold under the names Eastman AQ polymer (AQ35S, AQ38S, AQ55S, AQ48 Ultra) by the company Eastman Chemical.

Emulsions with no emulsifiers, stabilised by silicone particles or particles of metal oxide such as TiO₂ or the like, coated or uncoated, can also be prepared.

In a known manner, the composition of the invention can also contain additives such as hydrophilic or lipophilic gelling agents, hydrophobic or hydrophilic active substances, preservatives (for example phenoxyethanol and parabens), antioxidants, solvents, perfumes, fillers, bactericides, odour absorbers, colourant materials, and pH adjusters (acid or base or buffer). The quantities of these different additives are those normally used in the field in question, and for example are from 0.01 to 20% of the total weight of the composition. Depending on their nature, these additives can be introduced in the oily phase, in the aqueous phase, or dissolved in the surfactants.

Of course, the person skilled in the art will take care to select any additive or additives to add to the composition according to the invention, and the quantities thereof, in such a manner that the advantageous properties intrinsically attaching to the to the composition according to the invention are not, or essentially not, impaired by the intended addition.

According to a particular mode of implementation of the invention, the composition contains at least one hydrophilic, in other words water-soluble or water-dispersible, polymer. As hydrophilic polymers, the following may in particular be cited:
cellulose derivatives (carboxymethylcellulose, hydroxyethylcellulose and hydroxypropylmethylcellulose);
natural gums such as xanthan, guar or carob gums, and the carrageenans;
polycarboxyvinyl polymers of the Carbomer type, such as those sold by the company Goodrich under the names Carbopol 940, 951, 980, or by the company 3V-Sigma under the name Synthalen K or Synthalen L;
acrylic copolymers such as the copolymers of acrylates/alkylacrylates sold under the names Pemulen by the company Goodrich;
polyacrylamides and copolymers of acrylamide, indicated above, such as the product sold under the name of SEPIGEL 305 by the company Seppic, the product sold under the name of HOSTACERIN AMPS by the company Clariant or the copolymers sold under the names ARISTOFLEX by the company Clariant.

The quantity of hydrophilic polymer(s) can for example range from 0.01 to 5% by weight, preferably from 0.05 to 5% by weight and better from 0.1 to 3% by weight relative to the total weight of the composition.

Moreover, the composition can also contain at least one water-soluble hydroxyl compound selected from the C2-C6 and preferably C2-C4 monohydric alcohols such as ethanol and isopropanol, and polyols containing from 1 to 3 carbon atoms, such as glycerine, propylene glycol, butylene glycol, dipropylene glycol, isopropylene glycol and mixtures thereof. The quantity of hydroxyl compound(s) can for example range from 0.1 to 70% by weight, better from 1 to 65% by weight and still better from 1 to 60% by weight relative to the total weight of the composition.

When the composition is hydroalcoholic, it contains water and at least one monohydric alcohol, and when the composition is hydroglycolic, it contains water and at least one polyol. It can of course simultaneously contain alcohols and polyols.

The composition can also contain any active substance, such as for example urea and hydroxyl derivatives thereof such as the N-(2-hydroxyethyl)-urea marketed under the name hydrovance by the company National Starch, hyaluronic acid, hydrating polymers such as acrylic polymers with a phosphorylcholine group such as:
the 40% poly-2-(methacryloyloxyethyl)phosphoryl-choline in water/butanediol mixture (5% of butanediol) sold under the name LIPIDURE HM by the company Nippon Oils and Fats (INCI name: polyphosphorylcholine glycol acrylate (and) butylene glycol).
the 5% 2-(methacryloyloxyethyl)phosphorylcholine/butyl methacrylate (90/10) copolymer solution in water sold under the name LIPIDURE PMB by the company Nippon Oils and Fats; (INCI name: Polyquaternium-51),
the 5% 2-(methacryloyloxyethyl)phosphorylcholine/2-hydroxy-3-methacryloyloxypropyltrimethylammonium chloride copolymer solution in water, sold under the name LIPIDURE-C by the company Nippon Oils and Fats,
the 5% 2-(methacryloyloxyethyl)phosphorylcholine/butyl methacrylate/sodium methacrylate terpolymer solution in water, sold under the name LIPIDURE-A by the company Nippon Oils and Fats, and
the 2-(methacryloyloxyethyl) phosphorylcholine/stearyl methacrylate copolymers sold under the names LIPIDURE-S, LIPIDURE-NR and LIPIDURE-NA by the company Nippon Oils and Fats (INCI name: Polyquaternium 61).

The composition can also contain other active substances such as vitamins such as vitamins A, C, E, B3, B5 and K and derivatives thereof, in particular esters thereof, and sequestrants such as EDTA.

As fillers, the composition of the invention can for example contain mineral particles such as clays, silicas, metal oxides such as titanium dioxide or zinc oxide, mica and/or organic fillers such as polyamide (Nylon) particles and in particular those sold under the names ORGASOL by the company Atochem, latexes, polyethylene powders, microspheres based on acrylic copolymers, such as those of ethylene glycol dimethacrylate/lauryl methacrylate copolymer sold by the company Dow Corning under the name of POLYTRAP, microspheres of polymethyl methacrylate, marketed under the name MICROSPHERE M-100 by the company Matsumoto or under the name COVABEAD LH85 by the company Wackherr; ethylene-acrylate copolymer powders, such as those marketed under the name FLOBEADS by the company Sumitomo Seika Chemicals; expanded powders such as hollow microspheres and in particular, the microspheres formed of a vinylidene chloride, acrylonitrile and methacrylate terpolymer and marketed under the name EXPANCEL by the company Kemanord Plast, powders of natural organic materials such as starch powders, in particular maize, wheat or rice powders, whether or not crosslinked, such as the starch powders crosslinked with octenylsuccinic anhydride, marketed under the name DRY-FLO by the company National Starch, silicone resin microbeads such as those marketed under the name TOSPEARL by the company Toshiba Silicone, in particular TOSPEARL 240, and mixtures thereof. The quantity of filler(s) can for example range from 0.05 to 20% by weight and better 0.1 to 10% by weight relative to the total weight of the composition.

As previously indicated, this composition is preferably intended for use in a peeling process for the purpose of attenuating visible and/or tactile irregularities of the skin, and in particular for attenuating wrinkles and minor wrinkles and/or pigment spots and/or scars, in particular the marks from acne and/or to clear the pores of the skin and impart more lustre to the skin. The composition can therefore be applied in particular to the face and/or the neck and/or the neck and shoulders and/or the hands and/or the back.

In order to optimize its effects, the peeling process according to the invention preferably includes additional stages of preparation of the skin for the peeling and/or care for the skin after peeling by means of compositions containing lower quantities of active substances than the peeling composition described above.

The implementation of the above preliminary stage also makes it possible to detect any allergy to the active substances and to improve the effectiveness and the uniformity of the peeling.

Thus, according to a particular implementation mode, the process according to the invention includes, in addition to the stages mentioned above:
a preliminary stage of application to the skin of a preliminary composition comprising, in a physiologically acceptable medium, either from 1 to 15% by weight of one or more surfactants containing at least one alkyl chain having from 6 to 16 carbon atoms, relative to the total weight of the composition, or one or more hydroxy acids or a mixture of one or more surfactants containing at least one alkyl chain having from 6 to 16 carbon atoms and one or more hydroxy acids, or both, before the implementation of stage (a), and/or
a supplementary stage of application to the skin of a supplementary composition containing, in a physiologically acceptable medium, either from 15 to 70% by weight of one or more surfactants containing at least one alkyl chain having from 6 to 16 carbon atoms, relative to the total weight of the composition, or one or more hydroxy acids or a mixture of one or more surfactants containing at least one alkyl chain having from 6 to 16 carbon atoms and of one or more hydroxy acids, after the implementation of stage (c), where in all instances the preliminary composition and supplementary composition are preferably different from the composition described above.

The compositions utilized in these preliminary and supplementary stages can be applied morning and evening, for example, possibly in combination with a composition intended to protect the skin against the effects of UV. The pretreatment composition can be applied for one to four weeks and the post-treatment composition for one day to eight weeks, for example.

The process according to the invention, including the possible preliminary and supplementary stages, can be implemented a single time or repeated up to five times, if necessary, the peeling sessions preferably being separated by one to eight weeks.

The invention will now be illustrated by the following non-limiting examples. In these examples, the quantities are indicated in percentage by weight of active substance:

| | | Exam- | | |
|---|---|---|---|---|
| Composition | Example 1 | ple 2 | Example 3 | Example 4 |
| PEG-6 capric/caprylic | 13 | — | — | — |
| glyceride (1) | | | | |
| Coco glucoside (2) | — | 13 | — | — |
| Coco betaine (3) | — | — | 20 | — |
| Lauryl ether sulphate (4) | — | — | — | 50 |
| Glycolic acid | 20 | | | — |
| Salicylic acid | — | 20 | — | — |
| Lactic acid | — | — | — | 9 |
| Citric acid | — | — | 10 | — |
| Ethanol | — | 20 | — | — |
| Propylene glycol | | | | 20 |
| Soda | — | qs pH 4.5 | — | qs pH 3.5 |
| Water | qsp 100% | qsp | qsp 100% | qsp 100% |
| | | 100% | | |

(1) SOFTIGEN 767 from the company Sasol (100% active substance content)
(2) 24.53% of PLANTACARE 818 UP from the company Cognis which contains 53% active substance
(3) 66.67% of DEHYTON AB 30 from the company Cognis which contains 30% active substance
(4) 71.43% of TEXAPON AOS 225 UP from the company Cognis which contains 70% active substance

### Procedure:

The surfactants of the invention were dissolved in water, or, when the surfactants were in aqueous solution, these solutions were simply diluted. The hydroxy acids were dissolved in the surfactant solution.

The compositions obtained were utilized according to the procedure described above.

### Example 5

| | |
|---|---|
| glycolic acid | 30% |
| coco-glucoside | 13% |
| (say 24.53% of PLANTACARE 818 UP) | |
| water | qsp 100% |

Example 5 has a pH less than 2.

An in vitro test was performed to determine the effectiveness of Example 5 according to the invention in comparison with a composition with no surfactant, containing 30% of glycolic acid and 70% of water. The number of horny cells liberated after application of the composition according to the invention was about 370/μl, whereas it was about 40/μl for the composition with no surfactant, which shows that the combination according to the invention has a much greater effectiveness than a composition from the prior art.

The above written description of the invention provides a manner and process of making and using it such that any person skilled in this art is enabled to make and use the same, this enablement being provided in particular for the subject matter of the appended claims, which make up a part of the original description and including a process for the treatment of visible and/or tactile irregularities of the human skin, comprising:

(a) applying topically onto the skin a composition comprising in a physiologically acceptable medium (i) at least one hydroxy acid selected from the α-hydroxy acids, β-hydroxy acids, α-keto acids, β-keto acids and mixtures thereof, and (ii) at least 5% by weight, relative to the total weight of the composition, of one or more surfactants containing an alkyl chain having from 6 to 16 carbon atoms, the total quantity of hydroxy acids and surfactants containing one alkyl chain having from 6 to 16 carbon atoms being at least 15% by weight relative to the total weight of the composition, and optionally:
(b) leaving the composition in contact with the skin for a sufficient time for the composition to act, and optionally:
(c) removing the composition by rinsing.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” “the following can for example be cited,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

What is claimed is:

1. A process for the treatment of human skin, comprising applying topically onto the skin a composition comprising, in a physiologically acceptable medium, (i) at least one hydroxy acid selected from the group consisting of α-hydroxy acids, β-hydroxy acids, α-keto acids, β-keto acids and mixtures thereof, and (ii) at least 5% by weight, relative to the total weight of the composition, of one or more surfactants comprising an alkyl chain having from 6 to 16 carbon atoms, wherein the total amount of hydroxy acids and surfactants comprising an alkyl chain having from 6 to 16 carbon atoms is at least 15% by weight relative to the total weight of the composition.

2. The process according to claim 1, wherein the composition comprises at least 15% by weight of water relative to the total weight of the composition.

3. The process according to claim 1, wherein the one or more surfactants comprising an alkyl chain having from 6 to 16 carbon atoms have an HLB greater than 8.

4. The process according to claim 1, wherein the one or more surfactants comprising an alkyl chain having from 6 to 16 carbon atoms is (are) selected from the nonionic, anionic, cationic, amphoteric or zwitterionic surfactants, and mixtures thereof.

5. The process according to claim 1, wherein the composition comprises at least one nonionic surfactant selected from the surfactants comprising a group selected from polyalkylene glycol groups, polyglycerol groups, sugar groups, and mixtures thereof.

6. The process according to claim 1, wherein the composition comprises at least one nonionic surfactant selected from the alkylpolyglucosides, esters of poly-ethylene glycol and acids containing at least one alkyl chain ranging from C6 to C16, derivatives of polyethylene glycol and mono-, di- and triglycerides of acid containing at least one alkyl chain ranging from C6 to C16, ethoxylated derivatives of esters of sorbitan and acid containing at least one alkyl chain ranging from C6 to C16, sugar esters containing at least one C6 to C16 alkyl chain, esters of polyglycerol and acid containing at least one C6 to C16 alkyl chain, polyglycerol ethers, and mixtures thereof.

7. The process according to claim 1, wherein the composition comprises at least one anionic surfactant selected from the surfactants comprising a group selected from the sulphate, sulphonate, phosphate and carboxylate groups, amino acid, and mixtures thereof.

8. The process according to claim 1, wherein the composition comprises at least one anionic surfactant selected from alkylsulphates, alkyl ether sulphates and salts thereof, monoalkyl and dialkyl esters of phosphoric acid and salts thereof, alkali salts of amino acids, alkyl ether carboxylates, and mixtures thereof.

9. The process according to claim 1, wherein the composition comprises at least one cationic surfactant selected from surfactants comprising a quaternary ammonium group.

10. The process according to claim 1, wherein the composition comprises at least one amphoteric or zwitterionic surfactant selected from surfactants containing a group selected from the amphoacetate, amphodiacetate, betaine and sultaine groups.

11. The process according to claim 1, wherein the composition comprises at least one amphoteric or zwitterionic surfactant selected from the alkylbetaines, alkylamidopropylbetaines, alkylamphoacetates, hydroxy-sultaines and mixtures thereof.

12. The process according to claim 1, wherein the quantity of surfactants comprising an alkyl chain having from 6 to 16 carbon atoms ranges from 5 to 70% by weight relative to the total weight of the composition.

13. The process according to claim 1, wherein the composition comprises at least one α-hydroxy acid selected from citric acid, lactic acid, glycolic acid, tartaric acid, malic acid, mandelic acid, methyllacetic acid, glucuronic acid, pyruvic acid, phenyllactic acid, gluconic acid, galacturonic acid, and mixtures thereof.

14. The process according to claim 1, wherein the composition comprises at least one β-hydroxy acid selected from salicylic acid compounds of the following formula (I) or a salt thereof: 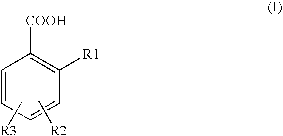
wherein:
R1 represents a hydroxy group or an ester of formula:
—O—CO—R₄
wherein R₄ is a saturated or unsaturated aliphatic group, containing from 1 to 26 carbon atoms, an amine or thiol group which may be substituted with an alkyl group containing from 1 to 18 carbon atoms,
R₂ and R₃ independently of one another are located in the 3, 4, 5 or 6 position on the benzene nucleus and independently of one another represent a hydrogen atom or a group:
—(O)ₙ—(CO)ₘ—R₅
wherein n and m, independently of one another, are each a whole number equal to 0 or 1, on condition that R₂ and R₃ are not simultaneously hydrogen atoms;
R₅ represents a hydrogen atom, a linear, branched or cyclized saturated aliphatic group containing from 1 to 18 carbon atoms, or an unsaturated group containing from 3 to 18 carbon atoms, bearing one to nine conjugated or non-conjugated double bonds, it being possible for the groups to be substituted with at least one substituent selected from halogen atoms, trifluoromethyl groups, hydroxyl groups in free form or esterified with an acid containing from 1 to 6 carbon atoms, or carboxyl groups, free or esterified with a lower alcohol containing from 1 to 6 carbon atoms.

15. The process according to claim 1, wherein the composition comprises at least one α-keto acid selected from ascorbic acid and ascorbyl glucosides.

16. The process according to claim 1, wherein the quantity of hydroxy acid(s) ranges from 5 to 70% by weight relative to the total weight of the composition.

17. The process according to claim 1, wherein the total quantity of hydroxy acids and surfactants containing an alkyl chain having from 6 to 16 carbon atoms is from 15 to 85% by weight relative to the total weight of the composition.

18. The process according to claim 1, wherein the composition has a pH of 1 to 9, preferably 1 to 5.

19. The process according to claim 1, wherein the composition is left in contact with the skin for a period of from 5 minutes to 12 hours.

20. The process according to claim 1, wherein it further comprises a preliminary stage of application onto the skin of a preliminary composition before application of said composition, said preliminary composition comprising, in a physiologically acceptable medium, either from 1 to 15% by weight of one or more surfactants comprising at least one alkyl chain having from 6 to 16 carbon atoms, relative to the total weight of the composition, or one or more hydroxy acids, or a mixture of one or more surfactants containing at least one alkyl chain having from 6 to 16 carbon atoms relative to the total weight of the composition and of one or more hydroxy acids, wherein said preliminary composition is different from said composition.

21. The process according to claim 1, wherein it further comprises removing said composition and, thereafter, the application to the skin of a supplementary composition comprising, in a physiologically acceptable medium, either from 15 to 70% by weight of one or more surfactants comprising at least one alkyl chain having from 6 to 16 carbon atoms, relative to the total weight of the composition, or one or more hydroxy acids, or a mixture of one or more surfactants comprising at least one alkyl chain having from 6 to 16 carbon atoms and of one or more hydroxy acids, wherein said composition and said supplementary composition are different.
